(19) United States
(12) Patent Application Publication
(10) Pub. No.: US 2008/0287866 A1
(43) Pub. Date: Nov. 20, 2008

(54) Methods and compositions for the treatment of pain

(76) Inventor: Adam Heller, Austin, TX (US)

Correspondence Address: CLARK & ELBING LLP, 101 FEDERAL STREET, BOSTON, MA, 02110, US

(21) Appl. No.: 12/012,115

(22) Filed: Jan. 31, 2008

Related U.S. Application Data
(60) Provisional application No. 60/887,431, filed on Jan. 31, 2007.
(60) Provisional application No. 60/930,261, filed on May 15, 2007.

(51) Int. Cl. A61K 31/131; A61P 25/00; A61M 31/00

(52) U.S. Cl. 604/82; 514/612

(57) ABSTRACT

The invention features methods, kits, and compositions for the treatment of pain.

### CROSS-REFERENCE TO RELATED APPLICATIONS

This application claims benefit of U.S. Provisional Ser. No. 60/887,431 filed on Jan. 31, 2007, and U.S. Provisional Ser. No. 60/930,261, filed on May 15, 2007, each of which is incorporated herein by reference.

### BACKGROUND OF THE INVENTION

Chronic pain is one of the most important clinical problems in all of medicine. For example, it is estimated that over 5 million people in the United States are disabled by back pain. The economic cost of chronic back pain is enormous, resulting in over 100 million lost work days annually at an estimated cost of $50-100 billion. It has been reported that approximately 8 million people in the U.S. report that they experience chronic neck or facial pain and spend an estimated $2 billion a year for treatment. The cost of managing pain for oncology patients is thought to approach $12 billion. Chronic pain disables more people than cancer or heart disease and costs the American public more than both cancer and heart disease combined. In addition to the physical consequences, chronic pain has numerous other costs including loss of employment, marital discord, depression and prescription drug addiction. It goes without saying, therefore, that reducing the morbidity and costs associated with persistent pain remains a significant challenge for the healthcare system.

Intractable severe pain resulting from injury, illness, scoliosis, spinal disc degeneration, spinal cord injury, malignancy, arachnoiditis, chronic disease, pain syndromes (e.g., failed back syndrome, complex regional pain syndrome) and other causes is a debilitating and common medical problem. In many patients, the continued use of analgesics, particularly drugs like narcotics, are not a viable solution due to tolerance, loss of effectiveness, and addiction potential.

Oxidizing agents are widely used as disinfectants. Examples of oxidizing disinfectants include aqueous solutions of chlorine, where hypochlorite and hypochlorous acid co-exist near neutral pH; of iodine, often dissolved as potassium tri-iodide in potassium iodide containing solutions; of hydrogen peroxide; of N-chloro-compounds, like Chloramine-T, the sodium salt of N-chloro-p-toluenesulfonamide and its salts with other cations or like Chloramine-B, the sodium salt of N-chloro-benzenesulfonamide and its salts with other cations; and of chloramine, NH2Cl, also known as monochloramine; and of ozone. All are bactericidal and/or fungicidal, and some also deactivate viruses. Chlorine and the product of its reaction with water hypochlorous acid, and chloramine, as well as ozone, are widely used to disinfect municipal drinking water. Oxidizing agents are also applied in sterilization, for example of surgical instruments.

Oxidizing agents are also used in treatment of wounds and disease. Bactericidal oxidizing agents are used also to disinfect wounds, to prevent and control pathogen-caused inflammation, to assist in the healing of skin and other wounds, and to treat pathogen-caused diseases. When used to disinfect wounds, they are optionally topically applied, for example by swabbing, brushing, spraying, or in a dressing. They are applied topically also in order to prevent infection before the skin is purposely pierced or cut, for example, prior to an injection, withdrawal of a blood sample, or surgery.

It is an object of the invention to provide new methods, kits, and compositions for the treatment of pain and itch.

### SUMMARY OF THE INVENTION

Applicants have discovered that oxidizing amines, such as N-chloro amines and N-dichloro amines, are useful for the treatment of pain and itch.

Accordingly, in a first aspect the invention features a method of treating pain in a patient in need thereof by topically administering to the patient an agent selected from N-chloro amines and N-dichloro amines in an amount sufficient to treat the pain.

The invention also features a method of treating pain at a site in a patient in need thereof by locally injecting at the site an agent selected from N-chloro amines and N-dichloro amines in an amount sufficient to treat the pain.

The invention further features a method of treating pain in a patient in need thereof by administering to the patient an agent selected from N-chloro amines and N-dichloro amines in an amount sufficient to treat the pain, wherein the pain is nociceptive pain, somatic pain, visceral pain, procedural pain, or inflammatory pain caused by trauma, surgery, or an autoimmune disease. In certain embodiments the pain is caused by trauma, surgery, herniation of an intervertebral disk, spinal cord injury, shingles, HIV/AIDS, cancer related pain, amputation, neurodegenerative disorders, carpal tunnel syndrome, diabetic neuropathy, postherpetic neuralgia, fibromyalgia, a musculoskeletal disorder, or any other painful condition described herein.

In a related aspect, the invention features a method of treating itch in a patient in need thereof by topically administering to the patient an agent selected from N-chloro amines and N-dichloro amines in an amount sufficient to treat the itch.

In an embodiment of any of the above aspects, the agent is administered locally at the site of pain or itch.

In another related aspect, the invention features a kit including (i) a composition including an agent selected from N-chloro amines and N-dichloro amines in an amount sufficient to treat pain when administered to a patient, and (ii) instructions for topically administering the composition to a patient for the treatment of pain. In certain embodiments, the composition is formulated for topical administration (e.g., formulated as a cream, lotion, spray, stick, iontophoresis solution, or ointment).

The invention also features a kit including (i) a composition formulated for injection and including an agent selected from N-chloro amines and N-dichloro amines in an amount sufficient to treat pain when administered to a patient, and (ii) instructions for locally injecting the composition at a site of a patient for the treatment of pain.

The invention further features a kit including (i) a composition including an agent selected from N-chloro amines and N-dichloro amines in an amount sufficient to treat pain when administered to a patient, and (ii) instructions for administering the composition to a patient for the treatment of nociceptive pain, somatic pain, visceral pain, procedural pain, or inflammatory pain caused by trauma, surgery, or an autoimmune disease. In certain embodiments, the kit further includes instructions for administering the composition to a patient suffering from pain caused by trauma, surgery, herniation of an intervertebral disk, spinal cord injury, shingles, HIV/AIDS, cancer related pain, amputation, neurodegenerative disorders, carpal tunnel syndrome, diabetic neuropathy, postherpetic neuralgia, fibromyalgia, a musculoskeletal disorder, or any other painful condition described herein.

The invention also features a kit including (i) a composition including an agent selected from N-chloro amines and N-dichloro amines in an amount sufficient to treat itch when administered to a patient, and (ii) instructions for topically administering the composition to a patient for the treatment of itch.

In a related aspect, the invention features a kit including (i) an inorganic oxide, (ii) an ammonium salt, (iii) a hypochlorite salt, (iv) instructions for contacting the inorganic oxide, the ammonium salt, and the hypochlorite salt with water to form a solution, and (v) instructions for administering the solution to a patient for the treatment of pain or itch. In certain embodiments, the kit further includes a buffer. In still other embodiments, the kit includes instructions for topically administering the solution into a patient for the treatment of pain or itch or instructions for infusing the solution into a patient at a site of pain.

In a related aspect, the invention features an infusion device including: (i) a first reservoir containing a first solution including an ammonium salt or an amine or a salt thereof; (ii) a second reservoir containing a second solution including hypochlorous acid or a salt thereof; (iii) a mixing chamber for combining the first solution and the second solution to form a chlorinated amine; and (iv) a cannula in fluid communication with the mixer chamber for delivering the N-chloro amine to a subject.

The invention further features an infusion device including: (i) a reservoir containing a first solution including an ammonium chloride salt and/or an amine and chloride ion; (ii) a power source electrically connected to an electrode in contact with the solution and configured to produce N-chloro amine via electrolysis; and (iii) a cannula in fluid communication with the solution for delivering the N-chloro amine to a subject.

The invention also features an infusion device including: (i) a first reservoir containing a first solution including an amine or a salt thereof; (ii) a second reservoir containing a second solution including a chloride ions; (iii) a power source electrically connected to an electrode in contact with the second solution and configured to produce hypochlorous acid or a salt thereof via electrolysis; (iv) a mixing chamber for combining the first solution and the hypochlorous acid or a salt thereof to form an N-chloro amine; and (v) a cannula in fluid communication with the mixing chamber for delivering the N-chloro amine to a subject.

In one embodiment of any of the above methods, kits, devices, or bandages the patient experiences some pain relief or some itch relief within 5, 10, 15, 20, 30, or 45 minutes of administering the chlorinated amine of the invention.

In another embodiment of any of the above methods, kits, devices, or bandages, the pain or itch does not result from an infection in the patient.

In yet another embodiment of any of the above methods, kits, devices, or bandages, the agent is selected from chloramine, chlorourea, N-chloro methylamine, N-chloro ethylamine, N-chloro isobutylamine, N-chloro-2-methylbutylamine, N-chloro pyrrolidine, N-chloro phenethylamine, N-chloro agmatine, N-chloro histamine, N-chloro tryptamine, N-chloro-3-methylthiopropanamine, N-chloro spermine, N-chloro carnosine, N-chloro carcinine, chloramine T, chloramine B, N-chloro glutathione sulfonamide, N-chloroglycine, N-chlorosulfamic acid, N-chlorosarcosine, N-chloro alpha-aminoisobutyric acid, N-chlorotaurine, N-chlorotaurine ethyl ester, N-chlorotaurine sulfonamide, N-chloro-acetylglycine, N-chloroalanine, N-chloro beta-alanine, N-chloro phenyl alanine, N-chloro norvaline, N-chloroleucine, N-chloro isoleucine, N-chloroproline, N-chloro omega aminoundecanoic acid, N-chloroaspartic acid, N-chloroglutamic acid, N-chloroasparagine, N-chlorovaline, N-chlorocystine, N-chloromethionine, N-chloroglutamine, N-chlorotryptophane, N-chlorohistidine, N-chloroarginine, N-chlorolysine, N-chloro alpha-aminobutyric acid, N-chloro gamma-aminobutyric acid, N-chloro alpha, epsilon diamino pimelic acid, N-chloro ornithine, N-chloroanthranilic acid, N-chloro p-aminobenzoic acid, N-chlorosulfanilic acid, N-chloro orthanilic acid, N-chloro phenyl sulfamic acid, N-chloroaminopropanesulfonic acid, N-chloro aminomethane-sulfonic, N-chloro glycylglycine, N-chloro glycylglycylglycine, N-chloro metanilic acid, N-chloro-N-octodecanyl glycine, dichloramine, N-dichloro methylamine, N-dichloro ethylamine, N-dichloro isobutylamine, N-dichloro-2-methylbutylamine, N-dichloro phenethylamine, N-dichloro agmatine, N-dichloro histamine, N-dichloro tryptamine, N-dichloro-3-methylthiopropanamine, N-dichloro spermine, N-dichloro carnosine, N-dichloro carcinine, N-dichloroglycine, N-dichloro alpha-aminoisobutyric acid, N-dichlorotaurine, N-dichlorotaurine ethyl ester, N-dichlorotaurine sulfonamide, N-dichloroalanine, N-dichloro beta-alanine, N-dichloro phenyl alanine, N-dichloro norvaline, N-dichloroleucine, N-dichloro isoleucine, N-dichloroproline, N-dichloro omega aminoundecanoic acid, N-dichloroaspartic acid, N-dichloroglutamic acid, N-dichloroasparagine, N-dichlorovaline, N-dichloromethionine, N-dichloroglutamine, N-dichlorotryptophane, N-dichloroarginine, N-dichlorolysine, N-dichloro alpha-aminobutyric acid, N-dichloro gamma-aminobutyric acid, N-dichloro alpha, epsilon diamino pimelic acid, N-dichloro ornithine, and pharmaceutically acceptable salts, esters, and amides thereof.

In certain embodiment of any of the above methods, kits, devices, or bandages, the agent is a chlorinated analgesic, chlorinated tricyclic antidepressant, chlorinated stimulant, or a polymer bearing N-chloro amine groups.

Chlorinated analgesics which can be used in the methods, kits, devices, and bandages of the invention include, without limitation, N-chloro lidocaine, desethyl-N-chloro lidocaine, N-chloro prilocalne, N-chloro tocainide, desethyl-N-chloro etidocaine, desbutyl-N-chloro ropivacaine, desbutyl-N-chloro bupivacaine, desbutyl-N-chloro levobupivacaine, desmethyl-N-chloro mepivacaine, desethyl-N-chloro procaine, desethyl-N-chloro proparacaine, desethyl-N-chloro allocain, desmethyl-N-chloro encainide, desethyl-N-chloro procainamide, desethyl-N-chloro metoclopramide, desmethyl-N-chloro stovaine, desethyl-N-chloro propoxycaine, desethyl-N-chloro chloroprocaine, N-chloro flecainide, desethyl-N-chloro tetracaine, N-chloro procaine, N-chloro proparacaine, N-chloro procainamide, N-chloro metoclopramide, N-chloro propoxycaine, N-chloro chloroprocaine, N-chloro tetracaine, N-chloro benzocaine, N-chloro butamben, and desethyl-N-chloro dibucaine.

Chlorinated tricyclic antidepressants which can be used in the methods, kits, devices, and bandages of the invention include, without limitation, N-chloro amoxapine, desmethyl-N-chloro trimipramine, desmethyl-N-chloro dothiepin, desmethyl-N-chloro doxepin, desmethyl-N-chloro amitriptyline, N-chloro protriptyline, N-chloro desipramine, desmethyl-N-chloro clomipramine, desmethyl-N-chloro clozapine, desmethyl-N-chloro loxapine, N-chloro nortriptyline, desmethyl-N-chloro cyclobenzaprine, desmethyl-N-chloro cyproheptadine, desmethyl-N-chloro olopatadine, desmethyl-N-chloro promethazine, desmethyl-N-chloro trimeprazine, desmethyl-N-chloro chlorprothixene, desmethyl-N-chloro chlorpromazine, desmethyl-N-chloro propiomazine, desmethyl-N-chloro prochlorperazine, desmethyl-N-chloro thiethylperazine, desmethyl-N-chloro trifluoperazine, desethyl-N-chloro ethacizine, and desmethyl-N-chloro imipramine.

Chlorinated stimulants which can be used in the methods, kits, devices, and bandages of the invention include, without limitation, N-chloro amphetamine, N-dichloro amphetamine, and N-chloro methamphetamine.

Polymers bearing N-chloro amine groups which can be used in the methods, kits, devices, and bandages of the invention include, without limitation, N-chlorinated chitosan, N-chlorinated deacetylated hyaluronic acid, and N-chlorinated polylysine.

In certain embodiment of any of the above methods, kits, devices, or bandages, the agent is selected from chloramine, dichloramine, N-chlorotaurine, N-dichloro taurine, N-chloro desmethylchlorpromazine, N-chloro lidocaine, N-chloro amphetamine, N-dichloro amphetamine, and N-chloro methamphetamine.

When used for topical administration, the chlorinated amine of the invention desirably has a solubility at about 25° C. of at least about 10⁻⁶ M, 10⁻⁵ M, 10⁻⁴ M, or 10⁻³ M both in water and in chloroform to allow for rapid diffusion to the nerve endings.

In certain embodiments the chlorinated amine of the invention has a long life (i.e., is highly stable) in comparison to chloramine, which has a half-life on the order of days. For example, sulfonate salts of N-chloro taurine may be desirable to use as such salts can be stored for many months without significant decomposition.

The term “pain” is used herein in the broadest sense and refers to all types of pain, including acute and chronic pain, such as nociceptive pain, e.g. somatic pain and visceral pain; inflammatory pain, dysfunctional pain, idiopathic pain, neuropathic pain, e.g., centrally generated pain and peripherally generated pain, migraine, and cancer pain.

The term “nociceptive pain” is used to include all pain caused by noxious stimuli that threaten to or actually injure body tissues, including, without limitation, by a cut, bruise, bone fracture, crush injury, and the like. Pain receptors for tissue injury (nociceptors) are located mostly in the skin, musculoskeletal system, or internal organs.

The term “somatic pain” is used to refer to pain arising from bone, joint, muscle, skin, or connective tissue. This type of pain is typically well localized.

The term “visceral pain” is used herein to refer to pain arising from visceral organs, such as the respiratory, gastrointestinal tract and pancreas, the urinary tract and reproductive organs. Visceral pain includes pain caused by tumor involvement of the organ capsule. Another type of visceral pain, which is typically caused by obstruction of hollow viscus, is characterized by intermittent cramping and poorly localized pain. Visceral pain may be associated with inflammation as in cystitis or reflux esophagitis.

The term “inflammatory pain” includes pain associated with active inflammation that may be caused by trauma, surgery, infection and autoimmune diseases.

The term “neuropathic pain” is used herein to refer to pain originating from abnormal processing of sensory input by the peripheral or central nervous system consequent on a lesion to these systems.

The term “procedural pain” refers to pain arising from a medical, dental or surgical procedure wherein the procedure is usually planned or associated with acute trauma.

The term “itch” (also known as pruritus) is used herein in the broadest sense and refers to all types of itching and stinging sensations localized and generalized, acute intermittent and persistent. The itch may be idiopathic, allergic, metabolic, drug-induced, due to liver, kidney disease, or cancer.

By “patient” is meant any animal. In one embodiment, the patient is a human. Other animals that can be treated using the methods and devices of the invention include but are not limited to non-human primates (e.g., monkeys, gorillas, chimpanzees), domesticated animals (e.g., horses, pigs, goats, rabbits, sheep, cattle, llamas), companion animals (e.g., guinea pigs, rats, mice, lizards, snakes, dogs, cats, fish, hamsters, and birds), animals participating in races or contests (horses, camels, dogs, birds), and marine mammals.

The term “pharmaceutically acceptable salt” represents those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. The salts can be prepared in situ during the final isolation and purification of the agents of the invention, or separately by reacting the free base function with a suitable organic acid. Representative acid addition salts include but are not limited to acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, isethionate, lactobionate, lactate, laurate, lauryl sulfate, malate, maleate, malonate, mesylate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include but are not limited to sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

As used herein, the terms “ester” and “amide” refer derivatives of the chlorinated amines described herein, including carboxylic acid esters and amides and sulfonic acid esters and amides. Examples of such derivatives include, without limitation the methyl, ethyl, isopropyl, propyl, butyl, and hexyl sulfonic acid esters of N-chloro taurine and N-dichloro taurine; sulfonamides of N-chloro taurine and N-dichloro taurine (e.g., formed from ammonia, secondary amines, or primary amines); methyl, ethyl, isopropyl, propyl, butyl, and hexyl carboxylic acid esters of amino acids, such as gamma aminobutyric acid; and carboxylic acid amides (e.g., formed from ammonia, secondary amines, or primary amines) of amino acids, such as gamma aminobutyric acid. Methods for making such derivatives are well known in the art.

By “treating pain” is meant preventing, reducing, or eliminating the sensation of pain in a subject. To treat pain, according to the methods of this invention, the treatment does not necessarily provide therapy for the underlying pathology that is causing the painful sensation. Treatment of pain can be purely symptomatic.

By “treating itch” is meant preventing, reducing, or eliminating the sensation of itch in a subject. To treat itch, according to the methods of this invention, the treatment does not necessarily provide therapy for the underlying pathology that is causing the itch. Treatment of itch can be purely symptomatic.

By “an amount sufficient” is meant an amount of an agent administered in a method of the invention required to prevent, reduce, or eliminate the sensation of pain (nociception) or itch. The effective amount of agent used to practice the present invention for therapeutic treatment of pain or itch varies depending upon the manner of administration, the age, and body weight, of the subject as well as the route of administration and underlying pathology that is causing the pain or itch. Ultimately, the attending physician or veterinarian will decide the appropriate amount and dosage regimen. Such amount is referred to as a “sufficient” amount.

By “chlorinated” is meant a material or compound bearing one or more N—H moieties which has been converted to an N—Cl moiety. For compounds and materials in which more than one nitrogen center can be chlorinated, the designation “N-chloro” refers to chlorination of at least one of the N—H moities. In contrast, the designation “N-dichloro” refers to a compound or material bearing at least one —NCl₂ moiety.

As used herein, “N-chloro-GSA” or “N-chloro-glutathione sulfonamide” refers specifically to chlorination of the sulfonamide nitrogen, as shown in the structure below.

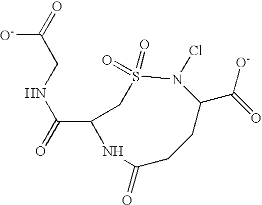

By “musculoskeletal disorder” is meant an immune system-related disorder of the muscles, ligaments, bones, joints, cartilage, or other connective tissue. Among the most commonly-occurring musculoskeletal disorders are various forms of arthritis, e.g., osteoarthritis, rheumatoid arthritis, juvenile rheumatoid arthritis, and gout. Other musculoskeletal disorders include acquired hyperostosis syndrome, acromegaly, ankylosing spondylitis, Behcet's disease, bone diseases, bursitis, cartilage diseases, chronic fatigue syndrome, compartment syndromes, congenital hypothyroidism, congenital myopathies, dentigerous cyst, dermatomyositis, diffuse idiopathic skeletal hyperostosis, Dupuytren's contracture, eosinophilia-myalgia syndrome, fasciitis, Felty's syndrome, fibromyalgia, hallux valgus, infectious arthritis, joint diseases, Kabuki make-up syndrome, Legg-Perthes disease, lupus, Lyme disease, Melas syndrome, metabolic bone diseases, mitochondrial myopathies, mixed connective tissue disease, muscular diseases, muscular dystrophies, musculoskeletal abnormalities, musculoskeletal diseases, myositis, myositis ossificans, necrotizing fasciitis, neurogenic arthropathy, osteitis deformans, osteochondritis, osteomalacia, osteomyelitis, osteonecrosis, osteoporosis, Paget's disease, Pierre Robin syndrome, polymyalgia rheumatica, polymyositis, postpoliomyelitis syndrome, pseudogout, psoriatic arthritis, reactive arthritis, Reiter disease, relapsing polychondritis, renal osteodystrophy, rhabdomyolysis, rheumatic diseases, rheumatic fever, scleroderma, Sever's disease (calceneal apophysitis), Sjögren's syndrome, spinal diseases, spinal stenosis, Still's disease, synovitis, temporomandibular joint disorders, tendinopathy, tennis elbow, tenosynovitis, Tietze's syndrome, and Wegener's granulomatosis.

The term “administration” or “administering” refers to a method of giving a dosage of agent to a patient, where the method is, e.g., topical, oral, nasal, ocular, subcutaneous, intravenous, intraperitoneal, or intramuscular. The preferred method of administration can vary depending on various factors, e.g., the components of the composition being administered, site of the pain or itch, and its severity.

The term “injection” refers to bolus or infusion delivery of an agent via needle or cannula, for example, subcutaneously, intravenously, intraperitoneally, or intramuscularly.

As used herein, “topical administration” refers to application of an agent of the invention to the skin of a subject. Topical administration includes transdermal administration, such as by iontophoresis.

A used herein, “local” administration or administration “locally” refers to the delivery of an agent at, or adjacent to, the site of pain or itching, or near part of a nerve transmitting the pain or itching-signal. For example, local administration is typically within about 2 cm or less from the affected nerve ending, preferably within less than about 0.5 cm and most preferably within less than about 2 mm from the ending.

Other features and advantages of the invention will be apparent from the following detailed description and the claims.

### DETAILED DESCRIPTION

We have discovered that chlorinated amines, such as chloramine, can be used to relieve pain and itch.

### Chlorinated Amines

The methods, kits, and compositions of the invention can include one or more N-chloro amines. The term “N-chloro amine” is a generic term meaning N-monochloro-compounds (e.g., Cl—NR₂, Cl—NHR, chlorinated amine-bearing polymers, N-chloro amides, N-chloro ureas, and N-chloro-sulfonamides). The term “N-dichloro amine” is a generic term meaning N-dichloro-compounds (e.g., Cl₂—NR). The chlorinated amines of this disclosure are oxidants, for example, of glutathione in vivo and are useful for the treatment of pain and itch. The invention can be carried out using chlorinated simple amines, such as ammonia, methylamine, or ethylamine, or amines which in their unchlorinated form have additional therapeutic utility, such as an analgesic (e.g., N-chloro lidocaine, desethyl-N-chloro lidocaine, N-chloro prilocalne, N-chloro tocainide, desethyl-N-chloro etidocaine, desbutyl-N-chloro ropivacaine, desbutyl-N-chloro bupivacaine, desbutyl-N-chloro levobupivacaine, desmethyl-N-chloro mepivacaine, desethyl-N-chloro procaine, desethyl-N-chloro proparacaine, desethyl-N-chloro allocain, desmethyl-N-chloro encainide, desethyl-N-chloro procainamide, desethyl-N-chloro metoclopramide, desmethyl-N-chloro stovaine, desethyl-N-chloro propoxycaine, desethyl-N-chloro chloroprocaine, N-chloro flecainide, desethyl-N-chloro tetracaine, N-chloro procaine, N-chloro proparacaine, N-chloro procainamide, N-chloro metoclopramide, N-chloro propoxycaine, N-chloro chloroprocaine, N-chloro tetracaine, N-chloro benzocaine, N-chloro butamben, and desethyl-N-chloro dibucaine); tricyclic antidepressant (e.g., N-chloro amoxapine, desmethyl-N-chloro trimipramine, desmethyl-N-chloro dothiepin, desmethyl-N-chloro doxepin, desmethyl-N-chloro amitriptyline, N-chloro protriptyline, N-chloro desipramine, desmethyl-N-chloro clomipramine, desmethyl-N-chloro clozapine, desmethyl-N-chloro loxapine, N-chloro nortriptyline, desmethyl-N-chloro cyclobenzaprine, desmethyl-N-chloro cyproheptadine, desmethyl-N-chloro olopatadine, desmethyl-N-chloro promethazine, desmethyl-N-chloro trimeprazine, desmethyl-N-chloro chlorprothixene, desmethyl-N-chloro chlorpromazine, desmethyl-N-chloro propiomazine, desmethyl-N-chloro prochlorperazine, desmethyl-N-chloro thiethylperazine, desmethyl-N-chloro trifluoperazine, desethyl-N-chloro ethacizine, or desmethyl-N-chloro imipramine), or stimulant (e.g., N-chloro amphetamine, N-dichloro amphetamine, or N-chloro methamphetamine). Other chlorinated amines that can be used in the methods, compositions, and kits of the invention are described below.

N-Chloro Amines

Exemplary N-chloro amines that can be used in the methods, kits, and compositions of the invention include, without limitation, chloramine, chlorourea, N-chloro methylamine, N-chloro ethylamine, N-chloro isobutylamine, N-chloro-2-methylbutylamine, N-chloro pyrrolidine, N-chloro phenethylamine, N-chloro agmatine, N-chloro histamine, N-chloro tryptamine, N-chloro-3-methylthiopropanamine, N-chloro spermine, N-chloro carnosine, N-chloro carcinine, chloramine T, chloramine B, N-chloro glutathione sulfonamide, N-chloroglycine, N-chlorosulfamic acid, N-chlorosarcosine, N-chloro alpha-aminoisobutyric acid, N-chlototaurine, N-chlorotaurine ethyl ester, N-chlorotaurine sulfonamide, N-chloro-acetylglycine, N-chloroalanine, N-chloro beta-alanine, N-chloro phenyl alanine, N-chloro norvaline, N-chloroleucine, N-chloro isoleucine, N-chloroproline, N-chloro omega aminoundecanoic acid, N-chloroaspartic acid, N-chloroglutamic acid, N-chloroasparagine, N-chlorovaline, N-chlorocystine, N-chloromethionine, N-chloroglutamine, N-chlorotryptophane, N-chlorohistidine, N-chloroarginine, N-chlorolysine, N-chloro alpha-aminobutyric acid, N-chloro gamma-aminobutyric acid, N-chloro alpha, epsilon diamino pimelic acid, N-chloro ornithine, N-chloroanthranilic acid, N-chloro p-aminobenzoic acid, N-chlorosulfanilic acid, N-chloro orthanilic acid, N-chloro phenyl sulfamic acid, N-chloroaminopropanesulfonic acid, N-chloro aminomethane-sulfonic, N-chloro glycylglycine, N-chloro glycylglycylglycine, N-chloro metanilic acid, and N-chloro-N-octodecanyl glycine.

Exemplary N-chloro-sulfonamides that can be used in the methods, kits, and compositions of the invention include, without limitation, N-chloro-GSA, Chloramine-B and Chloramine-T. N-chloro-sulfonamides can be used in their un-ionized and anionic forms. When anionic, it can be the free anion, or a salt, such as a Li⁺, Na⁺, K⁺, Ca²⁺, Mg²⁺, or Zn²⁺ salt.

Chlorinated Amine Polymers

The methods, kits, and compositions of the invention can include one or more chlorinated amine-bearing polymers. Exemplary chlorinated polymers which can be used in the methods, kits, and compositions of the invention include, without limitation, N-chlorinated chitosan, N-chlorinated deacetylated hyaluronic acid, and N-chlorinated polylysine. N-chlorinated amine-bearing polymers can be prepared using methods analogous to those described in U.S. Pat. No. 5,773,608, incorporated herein by reference.

N-Dichloro Amines

The methods, kits, and compositions of the invention can include one or more N-dichloro amines. Exemplary N-chloro amines that can be used in the methods, kits, and compositions of the invention include, without limitation, dichloramine, N-dichloro methylamine, N-dichloro ethylamine, N-dichloro isobutylamine, N-dichloro-2-methylbutylamine, N-dichloro phenethylamine, N-dichloro agmatine, N-dichloro histamine, N-dichloro tryptamine, N-dichloro-3-methylthiopropanamine, N-dichloro spermine, N-dichloro carnosine, N-dichloro carcinine, N-dichloroglycine, N-dichloro alpha-aminoisobutyric acid, N-dichlorotaurine, N-dichlorotaurine ethyl ester, N-dichlorotaurine sulfonamide, N-dichloroalanine, N-dichloro beta-alanine, N-dichloro phenyl alanine, N-dichloro norvaline, N-dichloroleucine, N-dichloro isoleucine, N-dichloroproline, N-dichloro omega aminoundecanoic acid, N-dichloroaspartic acid, N-dichloroglutamic acid, N-dichloroasparagine, N-dichlorovaline, N-dichloromethionine, N-dichloroglutamine, N-dichlorotryptophane, N-dichloroarginine, N-dichlorolysine, N-dichloro alpha-aminobutyric acid, N-dichloro gamma-aminobutyric acid, N-dichloro alpha, epsilon diamino pimelic acid, and N-dichloro ornithine.

Preparation of Chlorinated Amines

Chlorinated amines can be prepared by the reaction of the amine with a chlorine source under reaction conditions which lead to the replacement of one or two hydrogen atoms at the amino nitrogen with chlorine atoms. Such reactions are known to chemists skilled in the art. For example, the following chlorine sources, without limitation, may be used to produce the N-dichloro amines: HOCl or its salts (for example, NaOCl or KOCl), N-chloroarylsulfonamide salts (i.e., N-chloro-4-alkylbenzenesulfonamide); HClO₂, N-chloro-succinimide, Cl₂, thionylchloride, phosgene, PCl₃, PCl₅, and related chlorinating agents. In a typical reaction, the amine is dissolved in a lower alkanol (for example, methanol or ethanol) and made acidic. To this solution an aqueous NaOCl solution is added. The reaction results in the chlorination of the amino group and the precipitation of sodium chloride. The solvent is evaporated at low temperatures, for example, below 30° C. and a residue is obtained. The residue is taken up in a solvent and the chlorinated amine isolated, for example, by extraction with a solvent not miscible with the aqueous lower alkanol phase. The production of N-chloro versus N-dichloro derivatives can be controlled by the relative stoichiometry the amine and chlorinating agent. For additional experimental protocols see, for example, Marcinkiewicz et al., J. of Inflammatory Research 49:280 (2000); Chinake et al., Phys. Chem. Chem. Phys. 3:4957 (2001); Martincigh et al., J. Phys. Chem. A. 102:9838 (1998), and U.S. Pat. No. 3,932,605, each of which is incorporated herein by reference.

### Therapy and Formulation

The agents of the invention, N-chloro amines and N-dichloro amines, may be administered by any appropriate route for treatment of pain or itch. These may be administered to humans, domestic pets, livestock, or other animals with a pharmaceutically acceptable diluent, carrier, or excipient, in unit dosage form. Administration may be topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

Therapeutic formulations may be in the form of liquid solutions or suspensions; for oral administration, formulations may be in the form of tablets or capsules; and for intranasal formulations, in the form of powders, nasal drops, ear drops, or aerosols.

Methods well known in the art for making formulations are found, for example, in “Remington: The Science and Practice of Pharmacy” (20th ed., ed. A. R. Gennaro, 2000, Lippincott Williams & Wilkins). Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Nanoparticulate formulations (e.g., biodegradable nanoparticles, solid lipid nanoparticles, liposomes) may be used to control the biodistribution of the compounds. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycholate and deoxycholate, or may be oily solutions for administration in the form of nasal drops, or as a gel. The concentration of the compound in the formulation will vary depending upon a number of factors, including the dosage of the drug to be administered, and the route of administration.

The agents may be optionally administered as a pharmaceutically acceptable salt, such as a non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like.

Administration of compounds in controlled release formulations is useful where the compound of formula I has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastro-intestinal tract; or (iii) a short biological half-life, so that frequent dosing during a day is required in order to sustain the plasma level at a therapeutic level.

Many strategies can be pursued to obtain controlled release in which the rate of release outweighs the rate of metabolism of the therapeutic compound. For example, controlled release can be obtained by the appropriate selection of formulation parameters and ingredients, including, e.g., appropriate controlled release compositions and coatings. Examples include single or multiple unit tablet or capsule compositions, oil solutions, suspensions, emulsions, microcapsules, microspheres, nanoparticles, patches, and liposomes.

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

In accordance with the methods, kits, and compositions of the invention, agents can be infused into a patient using an infusion pump system. For example, skin-adhered infusion delivery systems which can be used include, without limitation, the pump systems described in U.S. Pat. Nos. 7,303,549; 7,303,543; 7,300,419; 7,297,138; 7,144,384; 7,070,580; 7,029,455; 7,018,360; 7,014,625; 6,960,192; 6,830,558; 6,768,425; 6,749,587; 6,740,059; 6,702,779; 6,699,218; 6,692,457; 6,669,669; 6,656,159; 6,656,158; 6,589,229; 6,520,938; 6,485,461; 6,475,196; 6,056,718 and 5,997,501, each of which is incorporated herein by reference. Existing insulin infusion pumps can be used to deliver an agent to a patient. Insulet Corporation of Bedford, Mass. manufactures and sells OmniPod, a small, lightweight self-adhesive insulin pod that the user fills with insulin and wears directly on the body for up to three days and then replaces. The OmniPod delivers precise, personalized doses of insulin into the body through a small subcutaneously inserted flexible cannula. The company also sells a wireless, handheld device that programs the OmniPod with the user's personalized insulin delivery instructions, wirelessly monitors the OmniPod's operation and incorporates a calibration device. The insulin solution typically employed with these devices can be replaced with an agent of the invention to deliver pain relief at the site of infusion. The dose-rate of agent is between about 10⁻⁹ moles per hour and about 10⁻⁴ moles per hour, preferably between about 10⁻⁸ moles per hour and about 10⁻⁵ moles per hour.

To relieve pain or itch a preferably aqueous solution or a gel including an agent of the invention is applied topically, for example in a dressing. The concentration of the agent in the solution or gel of the dressing is generally higher than about 0.1 mM and less than about 0.1 M. Preferably, it is higher than about 1 mM and is less than about 30 mM. Where the agent of the invention is gaseous, the dressing has a flexible shell that reduces at least tenfold, preferably one hundred fold, the out diffusion of gases other than hydrogen, for example of oxygen, chlorine, or chloramine. The shell can be adhered to the skin at its edges to form an adequately gas-tight seal slowing the leakage of the volatile agent. The shell can be made of a metallized, for example aluminized, plastic; or it can be made of a plastic through which gases permeate slowly, comprising, for example, polyvinylidene chloride, used in Saran™ wrap to retard evaporation of water and other volatile components of food.

Where the agents of the invention have a short half-life, the agent can be prepared just prior to administration. For example, a dry powder including an ammonium salt, chlorate salt, and metal oxide can be mixed with water to produce an N-chloro amine just prior to use.

In certain embodiments, the agents of the invention are adsorbed onto dry carrier particles, such as particles of talcum or zinc oxide, for gradual release when applied topically. In general the weight percentage of the adsorbed agent is at least about 0.01 wt % and is less than about 10 wt %; it is preferably at least about 0.1 wt % and is less than about 2 wt %. In general the weight percentage of the chemisorbed oxidant is at least about 0.01 wt % and is less than about 10 wt %; it is preferably at least about 0.1 wt % and is less than about 2 wt %.

The agents of the invention can also be delivered topically by iontophoresis. Iontophoresis is a needle-free, non-invasive technology for delivering bioactive agents through the skin using a small electric current to apply an electromotive force that transports ions through the stratum corneum, the outermost layer of skin, and into the dermis, the inner layer of skin that is comprised of connective tissue, blood and lymph vessels, sweat glands, hair follicles and an elaborate sensory nerve network.

### Indications

The methods, compositions, and kits of the invention are useful for treating pain, including clinical pain, namely inflammatory pain, functional pain, nociceptive pain, and neuropathic pain (e.g., peripheral neuropathic pain), whether acute or chronic (e.g., pain lasting for greater than one, two, three, four, or more months). Conditions that may be associated with pain include, for example, soft tissue, joint, bone inflammation and/or damage (e.g., acute trauma, osteoarthritis, or rheumatoid arthritis), myofascial pain syndromes (fibromylagia), headaches (including cluster headache, migraine and tension type headache), neurodegenerative disorders (i.e., particularly those leading to nerve demyelination), stump pain, myocardial infarction, angina, ischemic cardiovascular disease, post-stroke pain, sickle cell anemia, peripheral vascular occlusive disease, cancer, inflammatory conditions of the skin or joints, diabetic neuropathy, and acute tissue damage from surgery or traumatic injury (e.g., lacerations or fractures). The present invention is also useful for the treatment, reduction, or prevention of musculo-skeletal pain (after trauma or exercise), neuropathic pain caused by spinal cord injury, tumors, compression, inflammation, dental pain, episiotomy pain, deep and visceral pain (e.g., heart pain, bladder pain, or pelvic organ pain), muscle pain, eye pain, orofacial pain (e.g., odontalgia, trigeminal neuralgia, glossopharyngeal neuralgia), abdominal pain, gynecological pain (e.g., dysmenorrhea and labor pain), pain associated with nerve and root damage due to trauma, compression, inflammation, toxic chemicals, metabolic disorders, hereditary conditions, vasculitis and autoimmune diseases, central nervous system pain, such as pain due to spinal cord or brain stem damage, cerebrovascular accidents, tumors, infections, demyelinating diseases including multiple sclerosis, chronic lower back pain (e.g., ankylosing spondylitis, degenerative disk disease, radiculopathy, and radicular pain), sciatica, chronic neck pain, and post-operative pain (e.g., mastectomy, orthopedic and phantom limb pain). The present invention is also useful for treating pain associated with post-herpetic neuralgia, cancer, cystic fibrosis, HIV, and polymyalgia rheumatica. The methods, compositions, and kits of the invention can be used to treat pain associated with any of a number of conditions, including back and neck pain, cancer pain, gynecological and labor pain, fibromyalgia, arthritis and other rheumatological pains, orthopedic pains, post herpetic neuralgia and other neuropathic pains, sickle cell crises, interstitial cystitis, urethritis and other urological pains, dental pain, headaches, postoperative pain, and procedural pain (i.e., pain associated with injections, draining an abcess, surgery, dental procedures, opthalmic procedures, arthroscopies and use of other medical instrumentation, cosmetic surgical procedures, dermatological procedures, setting fractures, biopsies, and the like).

Pain and Function Indices

In order to measure the efficacy of any of the methods, compositions, or kits of the invention, a measurement index may be used. Indices that are useful in the methods, compositions, and kits of the invention for the measurement of pain associated with musculoskeletal, immunoinflammatory and neuropathic disorders include a visual analog scale (VAS), a Likert scale, categorical pain scales, descriptors, the Lequesne index, the WOMAC index, and the AUSCAN index, each of which is well known in the art. Such indices may be used to measure pain, itch, function, stiffness, or other variables.

A visual analog scale (VAS) provides a measure of a one-dimensional quantity. A VAS generally utilizes a representation of distance, such as a picture of a line with hash marks drawn at regular distance intervals, e.g., ten 1-cm intervals. For example, a patient can be asked to rank a sensation of pain or itch by choosing the spot on the line that best corresponds to the sensation of pain or itch, where one end of the line corresponds to “no pain” (score of 0 cm) or “no itch” and the other end of the line corresponds to “unbearable pain” or “unbearable itch” (score of 10 cm). This procedure provides a simple and rapid approach to obtaining quantitative information about how the patient is experiencing pain or itch. VAS scales and their use are described, e.g., in U.S. Pat. Nos. 6,709,406 and 6,432,937.

A Likert scale similarly provides a measure of a one-dimensional quantity. Generally, a Likert scale has discrete integer values ranging from a low value (e.g., 0, meaning no pain) to a high value (e.g., 7, meaning extreme pain). A patient experiencing pain is asked to choose a number between the low value and the high value to represent the degree of pain experienced. Likert scales and their use are described, e.g., in U.S. Pat. Nos. 6,623,040 and 6,766,319.

The Lequesne index and the Western Ontario and McMaster Universities (WOMAC) osteoarthritis index assess pain, function, and stiffness in the knee and hip of OA patients using self-administered questionnaires. Both knee and hip are encompassed by the WOMAC, whereas there is one Lequesne questionnaire for the knee and a separate one for the hip. These questionnaires are useful because they contain more information content in comparison with VAS or Likert. Both the WOMAC index and the Lequesne index questionnaires have been extensively validated in OA, including in surgical settings (e.g., knee and hip arthroplasty). Their metric characteristics do not differ significantly.

The AUSCAN (Australian-Canadian hand arthritis) index employs a valid, reliable, and responsive patient self-reported questionnaire. In one instance, this questionnaire contains 15 questions within three dimensions (Pain, 5 questions; Stiffness, 1 question; and Physical function, 9 questions). An AUSCAN index may utilize, e.g., a Likert or a VAS scale.

Indices that are useful in the methods, compositions, and kits of the invention for the measurement of pain include the Pain Descriptor Scale (PDS), the Visual Analog Scale (VAS), the Verbal Descriptor Scales (VDS), the Numeric Pain Intensity Scale (NPIS), the Neuropathic Pain Scale (NPS), the Neuropathic Pain Symptom Inventory (NPSI), the Present Pain Inventory (PPI), the Geriatric Pain Measure (GPM), the McGill Pain Questionnaire (MPQ), mean pain intensity (Descriptor Differential Scale), numeric pain scale (NPS) global evaluation score (GES) the Short-Form McGill Pain Questionnaire, the Minnesota Multiphasic Personality Inventory, the Pain Profile and Multidimensional Pain Inventory, the Child Heath Questionnaire, and the Child Assessment Questionnaire.

Itch can be measured by subjective measures (VAS, Lickert, descriptors). Another approach is to measure scratch which is an objective correlate of itch using a vibration transducer or movement-sensitive meters.

The following examples are intended to illustrate the invention, and is not intended to limit it.

### EXAMPLES

### Example 1

### Preparation of a Chloramine Containing Solution

A solution of 0.15 M ammonium chloride was prepared and its pH was adjusted with concentrated ammonia and with concentrated hydrochloric acid to about 7.4. The solution was chilled to 4° C. A second solution was prepared by adjusting the pH of the 10-13 weight % sodium hypochlorite solution (Sigma Aldrich, Milwaukee, Wis., Catalog #425044) to about 7.4 with concentrated hydrochloric acid. 10 mL of the about 1.5 M hypochlorite-hypochlorous acid solution was added promptly after adjusting its pH to 100 mL of the stirred and chilled first solution of 0.15 M ammonium chloride. The resulting solution was kept refrigerated until it was used.

### Example 2

### Relief of Pain by Topical Application

About 20 mL of the solution of Example 1 were poured onto absorbent paper towel and the wet towel was held for 10 min at a pain-causing wound in the skin of a finger of the inventor. The topical application of the wet paper towel caused mild, not unpleasant, local numbness and completely relieved the pain. After about 20 minutes there was neither numbness nor pain.

### Example 3

### Relief of Pain by Topical Application

As in Example 2, except that the site of pain was near the fingernail of an adult subject related to the inventor.

### Example 4

### Relief of Itching by Topical Application

As in Example 1, except that the wet paper towel was held against a zone of multiple, small, red, itching, scab-wounds on the skin of the lower leg of an adult subject related to the inventor. The itching was relieved for about 4 hours.

### Example 5

### Reduced Heat Sensitivity

To assess heat sensitivity, boiling water was poured into glasses and the inventor and an adult subject related to the inventor monitored the period for which they could tolerate pressing their chloramine-solution treated finger against the cup. They immersed their left index finger in the chloramine solution, prepared as described in Example 1, now at ambient temperature, and their right index fingers into a tap water solution for 15 minutes then pressed their fingers against the hot cups. They were able to press their chloramine-solution treated fingers considerably longer, typically for periods between about 1.5 times and twice as long as they could press their tap-water treated fingers.

### Example 6

### Painful Blisters

An adult subject presented with painful blisters of unknown origin on the little finger of her left hand. The subject had a history of diagnosed eczema on her face and undiagnosed but likely eczema on her hands. In previous suspected eczema episodes on her hands blisters have formed, but more generally on the hands and never in a cluster such as this. Prior to treatment, the whole fingertip hurt and throbbed, and the joint was painful to bend due to pressure on the blisters.

Fresh chloramine solution was prepared as described in Example 1.

In a first test, the subject dipped her finger into a cold chloramine solution for 10 minutes and reported immediate pain relief, absence of throbbing and the ability to bend the joint without pain. When she touched the blisters directly there was a feeling of “pressure,” but not pain. The effect lasted for about two hours and the pain had fully returned after three hours.

In a second test, very cold water was substituted for the chloramine solution. The subject dipped her finger into the water for ten minutes and reported that the blisters were still “quite painful” to the touch.

In a third test, the original chloramine solution was applied again and the subject again reported pain relief.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A method of treating pain in a patient in need thereof, said method comprising topically administering to said patient an agent selected from N-chloro amines and N-dichloro amines in an amount sufficient to treat said pain.

2. A method of treating pain at a site in a patient in need thereof, said method comprising locally injecting at said site an agent selected from N-chloro amines and N-dichloro amines in an amount sufficient to treat said pain.

3. A method of treating pain in a patient in need thereof, said method comprising administering to said patient an agent selected from N-chloro amines and N-dichloro amines in an amount sufficient to treat said pain, wherein said pain is nociceptive pain, somatic pain, visceral pain, procedural pain, or inflammatory pain caused by trauma, surgery, or an autoimmune disease.

4. The method of claim 3, wherein said pain is caused by trauma, surgery, herniation of an intervertebral disk, spinal cord injury, shingles, HIV/AIDS, cancer related pain, amputation, neurodegenerative disorders, carpal tunnel syndrome, diabetic neuropathy, postherpetic neuralgia, fibromyalgia, or a musculoskeletal disorder.

5. A method of treating itch in a patient in need thereof, said method comprising topically administering to said patient an agent selected from N-chloro amines and N-dichloro amines in an amount sufficient to treat said itch.

6. The method of any of claims 1, 3, or 5, wherein said agent is administered locally at the site of pain or itch.

7. The method of any of claims 1, 3, or 5, wherein said agent is selected from chloramine, chlorourea, N-chloro methylamine, N-chloro ethylamine, N-chloro isobutylamine, N-chloro-2-methylbutylamine, N-chloro pyrrolidine, N-chloro phenethylamine, N-chloro agmatine, N-chloro histamine, N-chloro tryptamine, N-chloro-3-methylthiopropanamine, N-chloro spermine, N-chloro carnosine, N-chloro carcinine, chloramine T, chloramine B, N-chloro glutathione sulfonamide, N-chloroglycine, N-chlorosulfamic acid, N-chlorosarcosine, N-chloro alpha-aminoisobutyric acid, N-chlorotaurine, N-chlorotaurine ethyl ester, N-chlorotaurine sulfonamide, N-chloro-acetylglycine, N-chloroalanine, N-chloro beta-alanine, N-chloro phenyl alanine, N-chloro norvaline, N-chloroleucine, N-chloro isoleucine, N-chloroproline, N-chloro omega aminoundecanoic acid, N-chloroaspartic acid, N-chloroglutamic acid, N-chloroasparagine, N-chlorovaline, N-chlorocystine, N-chloromethionine, N-chloroglutamine, N-chlorotryptophane, N-chlorohistidine, N-chloroarginine, N-chlorolysine, N-chloro alpha-aminobutyric acid, N-chloro gamma-aminobutyric acid, N-chloro alpha, epsilon diamino pimelic acid, N-chloro ornithine, N-chloroanthranilic acid, N-chloro p-aminobenzoic acid, N-chlorosulfanilic acid, N-chloro orthanilic acid, N-chloro phenyl sulfamic acid, N-chloroaminopropanesulfonic acid, N-chloro aminomethane-sulfonic, N-chloro glycylglycine, N-chloro glycylglycylglycine, N-chloro metanilic acid, N-chloro-N-octodecanyl glycine, dichloramine, N-dichloro methylamine, N-dichloro ethylamine, N-dichloro isobutylamine, N-dichloro-2-methylbutylamine, N-dichloro phenethylamine, N-dichloro agmatine, N-dichloro histamine, N-dichloro tryptamine, N-dichloro-3-methylthiopropanamine, N-dichloro spermine, N-dichloro carnosine, N-dichloro carcinine, N-dichloroglycine, N-dichloro alpha-aminoisobutyric acid, N-dichlorotaurine, N-dichlorotaurine ethyl ester, N-dichlorotaurine sulfonamide, N-dichloroalanine, N-dichloro beta-alanine, N-dichloro phenyl alanine, N-dichloro norvaline, N-dichloroleucine, N-dichloro isoleucine, N-dichloroproline, N-dichloro omega aminoundecanoic acid, N-dichloroaspartic acid, N-dichloroglutamic acid, N-dichloroasparagine, N-dichlorovaline, N-dichloromethionine, N-dichloroglutamine, N-dichlorotryptophane, N-dichloroarginine, N-dichlorolysine, N-dichloro alpha-aminobutyric acid, N-dichloro gamma-aminobutyric acid, N-dichloro alpha, epsilon diamino pimelic acid, N-dichloro ornithine, and pharmaceutically acceptable salts, esters, and amides thereof.

8. The method of any of claims 1, 3, or 5, wherein said agent is a chlorinated analgesic, chlorinated tricyclic antidepressant, chlorinated stimulant, or a polymer bearing N-chloro amine groups.

9. The method of claim 8, wherein said agent is a chlorinated analgesic selected from N-chloro lidocaine, desethyl-N-chloro lidocaine, N-chloro prilocaine, N-chloro tocainide, desethyl-N-chloro etidocaine, desbutyl-N-chloro ropivacaine, desbutyl-N-chloro bupivacaine, desbutyl-N-chloro levobupivacaine, desmethyl-N-chloro mepivacaine, desethyl-N-chloro procaine, desethyl-N-chloro proparacaine, desethyl-N-chloro allocain, desmethyl-N-chloro encainide, desethyl-N-chloro procainamide, desethyl-N-chloro metoclopramide, desmethyl-N-chloro stovaine, desethyl-N-chloro propoxycaine, desethyl-N-chloro chloroprocaine, N-chloro flecainide, desethyl-N-chloro tetracaine, N-chloro procaine, N-chloro proparacaine, N-chloro procainamide, N-chloro metoclopramide, N-chloro propoxycaine, N-chloro chloroprocaine, N-chloro tetracaine, N-chloro benzocaine, N-chloro butamben, and desethyl-N-chloro dibucaine.

10. The method of claim 8, wherein said agent is a chlorinated tricyclic antidepressant selected from N-chloro amoxapine, desmethyl-N-chloro trimipramine, desmethyl-N-chloro dothiepin, desmethyl-N-chloro doxepin, desmethyl-N-chloro amitriptyline, N-chloro protriptyline, N-chloro desipramine, desmethyl-N-chloro clomipramine, desmethyl-N-chloro clozapine, desmethyl-N-chloro loxapine, N-chloro nortriptyline, desmethyl-N-chloro cyclobenzaprine, desmethyl-N-chloro cyproheptadine, desmethyl-N-chloro olopatadine, desmethyl-N-chloro promethazine, desmethyl-N-chloro trimeprazine, desmethyl-N-chloro chlorprothixene, desmethyl-N-chloro chlorpromazine, desmethyl-N-chloro propiomazine, desmethyl-N-chloro prochlorperazine, desmethyl-N-chloro thiethylperazine, desmethyl-N-chloro trifluoperazine, desethyl-N-chloro ethacizine, and desmethyl-N-chloro imipramine.

11. The method of claim 8, wherein said agent is a chlorinated stimulant selected from N-chloro amphetamine, N-dichloro amphetamine, and N-chloro methamphetamine.

12. The method of claim 8, wherein said agent is a polymer selected from N-chlorinated chitosan, N-chlorinated deacetylated hyaluronic acid, and N-chlorinated polylysine.

13. The method of any of claims 1, 3, or 5, wherein said agent is selected from chloramine, dichloramine, N-chlorotaurine, N-dichloro taurine, N-chloro desmethylchlorpromazine, N-chloro lidocaine, N-chloro amphetamine, N-dichloro amphetamine, and N-chloro methamphetamine.

14. The method of claim 1 or 3, wherein said patient experiences some pain relief within 20 minutes of administering said agent.

15. The method of claim 5, wherein said patient experiences some relief from itch within 20 minutes of administering said agent.

16. The method of claim 1 or 3, with the proviso that said pain does not result from an infection in said patient.

17. A kit comprising (i) a composition comprising an agent selected from N-chloro amines and N-dichloro amines in an amount sufficient to treat pain when administered to a patient, and (ii) instructions for topically administering said composition to a patient for the treatment of pain.

18. The kit of claim 17, wherein said composition is formulated for topical administration.

19. The kit of claim 18, wherein said composition is formulated as a cream, lotion, spray, stick, iontophoresis solution, or ointment.

20. A kit comprising (i) a composition formulated for injection and comprising an agent selected from N-chloro amines and N-dichloro amines in an amount sufficient to treat pain when administered to a patient, and (ii) instructions for locally injecting said composition at a site of a patient for the treatment of pain.

21. A kit comprising (i) a composition comprising an agent selected from N-chloro amines and N-dichloro amines in an amount sufficient to treat pain when administered to a patient, and (ii) instructions for administering said composition to a patient for the treatment of nociceptive pain, somatic pain, visceral pain, procedural pain, or inflammatory pain caused by trauma, surgery, or an autoimmune disease.

22. The kit of claim 21, further comprising instructions for administering said composition to a patient suffering from pain caused by trauma, surgery, herniation of an intervertebral disk, spinal cord injury, shingles, HIV/AIDS, cancer related pain, amputation, neurodegenerative disorders, carpal tunnel syndrome, diabetic neuropathy, postherpetic neuralgia, fibromyalgia, or a musculoskeletal disorder.

23. A kit comprising (i) a composition comprising an agent selected from N-chloro amines and N-dichloro amines in an amount sufficient to treat itch when administered to a patient, and (ii) instructions for topically administering said composition to a patient for the treatment of itch.

24. The kit of any of claims 17, 20, 21, or 23, wherein said agent is selected from chloramine, chlorourea, N-chloro methylamine, N-chloro ethylamine, N-chloro isobutylamine, N-chloro-2-methylbutylamine, N-chloro pyrrolidine, N-chloro phenethylamine, N-chloro agmatine, N-chloro histamine, N-chloro tryptamine, N-chloro-3-methylthiopropanamine, N-chloro spermine, N-chloro carnosine, N-chloro carcinine, chloramine T, chloramine B, N-chloro glutathione sulfonamide, N-chloroglycine, N-chlorosulfamic acid, N-chlorosarcosine, N-chloro alpha-aminoisobutyric acid, N-chlorotaurine, N-chlorotaurine ethyl ester, N-chlorotaurine sulfonamide, N-chloro-acetylglycine, N-chloroalanine, N-chloro beta-alanine, N-chloro phenyl alanine, N-chloro norvaline, N-chloroleucine, N-chloro isoleucine, N-chloroproline, N-chloro omega aminoundecanoic acid, N-chloroaspartic acid, N-chloroglutamic acid, N-chloroasparagine, N-chlorovaline, N-chlorocystine, N-chloromethionine, N-chloroglutamine, N-chlorotryptophane, N-chlorohistidine, N-chloroarginine, N-chlorolysine, N-chloro alpha-aminobutyric acid, N-chloro gamma-aminobutyric acid, N-chloro alpha, epsilon diamino pimelic acid, N-chloro ornithine, N-chloroanthranilic acid, N-chloro p-aminobenzoic acid, N-chlorosulfanilic acid, N-chloro orthanilic acid, N-chloro phenyl sulfamic acid, N-chloroaminopropanesulfonic acid, N-chloro aminomethane-sulfonic, N-chloro glycylglycine, N-chloro glycylglycylglycine, N-chloro metanilic acid, N-chloro-N-octodecanyl glycine, dichloramine, N-dichloro methylamine, N-dichloro ethylamine, N-dichloro isobutylamine, N-dichloro-2-methylbutylamine, N-dichloro phenethylamine, N-dichloro agmatine, N-dichloro histamine, N-dichloro tryptamine, N-dichloro-3-methylthiopropanamine, N-dichloro spermine, N-dichloro carnosine, N-dichloro carcinine, N-dichloroglycine, N-dichloro alpha-aminoisobutyric acid, N-dichlorotaurine, N-dichlorotaurine ethyl ester, N-dichlorotaurine sulfonamide, N-dichloroalanine, N-dichloro beta-alanine, N-dichloro phenyl alanine, N-dichloro norvaline, N-dichloroleucine, N-dichloro isoleucine, N-dichloroproline, N-dichloro omega aminoundecanoic acid, N-dichloroaspartic acid, N-dichloroglutamic acid, N-dichloroasparagine, N-dichlorovaline, N-dichloromethionine, N-dichloroglutamine, N-dichlorotryptophane, N-dichloroarginine, N-dichlorolysine, N-dichloro alpha-aminobutyric acid, N-dichloro gamma-aminobutyric acid, N-dichloro alpha, epsilon diamino pimelic acid, N-dichloro ornithine, and pharmaceutically acceptable salts, esters, and amides thereof.

25. The kit of any of claims 17, 20, 21, or 23, wherein said agent is a chlorinated analgesic, chlorinated tricyclic antidepressant, chlorinated stimulant, or a polymer bearing N-chloro amine groups.

26. The kit of claim 25, wherein said agent is a chlorinated analgesic selected from N-chloro lidocaine, desethyl-N-chloro lidocaine, N-chloro prilocalne, N-chloro tocainide, desethyl-N-chloro etidocaine, desbutyl-N-chloro ropivacaine, desbutyl-N-chloro bupivacaine, desbutyl-N-chloro levobupivacaine, desmethyl-N-chloro mepivacaine, desethyl-N-chloro procaine, desethyl-N-chloro proparacaine, desethyl-N-chloro allocain, desmethyl-N-chloro encainide, desethyl-N-chloro procainamide, desethyl-N-chloro metoclopramide, desmethyl-N-chloro stovaine, desethyl-N-chloro propoxycaine, desethyl-N-chloro chloroprocaine, N-chloro flecainide, desethyl-N-chloro tetracaine, N-chloro procaine, N-chloro proparacaine, N-chloro procainamide, N-chloro metoclopramide, N-chloro propoxycaine, N-chloro chloroprocaine, N-chloro tetracaine, N-chloro benzocaine, N-chloro butamben, and desethyl-N-chloro dibucaine.

27. The kit of claim 25, wherein said agent is a chlorinated tricyclic antidepressant selected from N-chloro amoxapine, desmethyl-N-chloro trimipramine, desmethyl-N-chloro dothiepin, desmethyl-N-chloro doxepin, desmethyl-N-chloro amitriptyline, N-chloro protriptyline, N-chloro desipramine, desmethyl-N-chloro clomipramine, desmethyl-N-chloro clozapine, desmethyl-N-chloro loxapine, N-chloro nortriptyline, desmethyl-N-chloro cyclobenzaprine, desmethyl-N-chloro cyproheptadine, desmethyl-N-chloro olopatadine, desmethyl-N-chloro promethazine, desmethyl-N-chloro trimeprazine, desmethyl-N-chloro chlorprothixene, desmethyl-N-chloro chlorpromazine, desmethyl-N-chloro propiomazine, desmethyl-N-chloro prochlorperazine, desmethyl-N-chloro thiethylperazine, desmethyl-N-chloro trifluoperazine, desethyl-N-chloro ethacizine, and desmethyl-N-chloro imipramine.

28. The kit of claim 25, wherein said agent is a chlorinated stimulant selected from N-chloro amphetamine, N-dichloro amphetamine, and N-chloro methamphetamine.

29. The kit of claim 25, wherein said agent is a polymer selected from N-chlorinated chitosan, N-chlorinated deacetylated hyaluronic acid, and N-chlorinated polylysine.

30. The kit of any of claims 17, 20, 21, or 23, wherein said agent is selected from chloramine, dichloramine, N-chloro taurine, N-dichloro taurine, N-chloro desmethylchlorpromazine, N-chloro lidocaine, N-chloro amphetamine, N-dichloro amphetamine, and N-chloro methamphetamine.

31. A kit comprising (i) an inorganic oxide, (ii) an ammonium salt, (iii) a hypochlorite salt, (iv) instructions for contacting said inorganic oxide, said ammonium salt, and said hypochlorite salt with water to form a solution, and (v) instructions for administering said solution to a patient for the treatment of pain or itch.

32. The kit of claim 31, further comprising a buffer.

33. The kit of claim 31, further comprising instructions for topically administering said solution into a patient for the treatment of pain or itch.

34. The kit of claim 31, further comprising instructions for infusing said solution into a patient at a site of pain.

35. A bandage comprising an agent selected from N-chloro amines and N-dichloro amines in an amount sufficient to treat pain or itch when applied to the skin of a patient.

36. The bandage of claim 35, wherein said agent is selected from chloramine, chlorourea, N-chloro methylamine, N-chloro ethylamine, N-chloro isobutylamine, N-chloro-2-methylbutylamine, N-chloro pyrrolidine, N-chloro phenethylamine, N-chloro agmatine, N-chloro histamine, N-chloro tryptamine, N-chloro-3-methylthiopropanamine, N-chloro spermine, N-chloro carnosine, N-chloro carcinine, chloramine T, chloramine B, N-chloro glutathione sulfonamide, N-chloroglycine, N-chlorosulfamic acid, N-chlorosarcosine, N-chloro alpha-aminoisobutyric acid, N-chlorotaurine, N-chlorotaurine ethyl ester, N-chlorotaurine sulfonamide, N-chloro-acetylglycine, N-chloroalanine, N-chloro beta-alanine, N-chloro phenyl alanine, N-chloro norvaline, N-chloroleucine, N-chloro isoleucine, N-chloroproline, N-chloro omega aminoundecanoic acid, N-chloroaspartic acid, N-chloroglutamic acid, N-chloroasparagine, N-chlorovaline, N-chlorocystine, N-chloromethionine, N-chloroglutamine, N-chlorotryptophane, N-chlorohistidine, N-chloroarginine, N-chlorolysine, N-chloro alpha-aminobutyric acid, N-chloro gamma-aminobutyric acid, N-chloro alpha, epsilon diamino pimelic acid, N-chloro ornithine, N-chloroanthranilic acid, N-chloro p-aminobenzoic acid, N-chlorosulfanilic acid, N-chloro orthanilic acid, N-chloro phenyl sulfamic acid, N-chloroaminopropanesulfonic acid, N-chloro aminomethane-sulfonic, N-chloro glycylglycine, N-chloro glycylglycylglycine, N-chloro metanilic acid, N-chloro-N-octodecanyl glycine, dichloramine, N-dichloro methylamine, N-dichloro ethylamine, N-dichloro isobutylamine, N-dichloro-2-methylbutylamine, N-dichloro phenethylamine, N-dichloro agmatine, N-dichloro histamine, N-dichloro tryptamine, N-dichloro-3-methylthiopropanamine, N-dichloro spermine, N-dichloro carnosine, N-dichloro carcinine, N-dichloroglycine, N-dichloro alpha-aminoisobutyric acid, N-dichlorotaurine, N-dichlorotaurine ethyl ester, N-dichlorotaurine sulfonamide, N-dichloroalanine, N-dichloro beta-alanine, N-dichloro phenyl alanine, N-dichloro norvaline, N-dichloroleucine, N-dichloro isoleucine, N-dichloroproline, N-dichloro omega aminoundecanoic acid, N-dichloroaspartic acid, N-dichloroglutamic acid, N-dichloroasparagine, N-dichlorovaline, N-dichloromethionine, N-dichloroglutamine, N-dichlorotryptophane, N-dichloroarginine, N-dichlorolysine, N-dichloro alpha-aminobutyric acid, N-dichloro gamma-aminobutyric acid, N-dichloro alpha, epsilon diamino pimelic acid, N-dichloro ornithine, and pharmaceutically acceptable salts, esters, and amides thereof.

37. The bandage of claim 35, wherein said agent is a chlorinated analgesic, chlorinated tricyclic antidepressant, chlorinated stimulant, or a polymer bearing N-chloro amine groups.

38. The bandage of claim 37, wherein said agent is a chlorinated analgesic selected from N-chloro lidocaine, desethyl-N-chloro lidocaine, N-chloro prilocalne, N-chloro tocainide, desethyl-N-chloro etidocaine, desbutyl-N-chloro ropivacaine, desbutyl-N-chloro bupivacaine, desbutyl-N-chloro levobupivacaine, desmethyl-N-chloro mepivacaine, desethyl-N-chloro procaine, desethyl-N-chloro proparacaine, desethyl-N-chloro allocain, desmethyl-N-chloro encainide, desethyl-N-chloro procainamide, desethyl-N-chloro metoclopramide, desmethyl-N-chloro stovaine, desethyl-N-chloro propoxycaine, desethyl-N-chloro chloroprocaine, N-chloro flecainide, desethyl-N-chloro tetracaine, N-chloro procaine, N-chloro proparacaine, N-chloro procainamide, N-chloro metoclopramide, N-chloro propoxycaine, N-chloro chloroprocaine, N-chloro tetracaine, N-chloro benzocaine, N-chloro butamben, and desethyl-N-chloro dibucaine.

39. The bandage of claim 37, wherein said agent is a chlorinated tricyclic antidepressant selected from N-chloro amoxapine, desmethyl-N-chloro trimipramine, desmethyl-N-chloro dothiepin, desmethyl-N-chloro doxepin, desmethyl-N-chloro amitriptyline, N-chloro protriptyline, N-chloro desipramine, desmethyl-N-chloro clomipramine, desmethyl-N-chloro clozapine, desmethyl-N-chloro loxapine, N-chloro nortriptyline, desmethyl-N-chloro cyclobenzaprine, desmethyl-N-chloro cyproheptadine, desmethyl-N-chloro olopatadine, desmethyl-N-chloro promethazine, desmethyl-N-chloro trimeprazine, desmethyl-N-chloro chlorprothixene, desmethyl-N-chloro chlorpromazine, desmethyl-N-chloro propiomazine, desmethyl-N-chloro prochlorperazine, desmethyl-N-chloro thiethylperazine, desmethyl-N-chloro trifluoperazine, desethyl-N-chloro ethacizine, and desmethyl-N-chloro imipramine.

40. The bandage of claim 37, wherein said agent is a chlorinated stimulant selected from N-chloro amphetamine, N-dichloro amphetamine, and N-chloro methamphetamine.

41. The bandage of claim 37, wherein said agent is a polymer selected from N-chlorinated chitosan, N-chlorinated deacetylated hyaluronic acid, and N-chlorinated polylysine.

42. The bandage of claim 35, wherein said agent is selected from chloramine, dichloramine, N-chloro taurine, N-dichloro taurine, N-chloro desmethylchlorpromazine, N-chloro lidocaine, N-chloro amphetamine, N-dichloro amphetamine, and N-chloro methamphetamine.

43. The bandage of claim 35, wherein a patient experiences some relief from pain or itch within 20 minutes of applying said bandage to the skin of said patient.

44. An infusion device comprising:
(i) a first reservoir containing a first solution comprising an ammonium salt or an amine or a salt thereof;
(ii) a second reservoir containing a second solution comprising hypochlorous acid or a salt thereof;
(iii) a mixing chamber for combining said first solution and said second solution to form a chlorinated amine; and
(iv) a cannula in fluid communication with said mixer chamber for delivering said N-chloro amine to a subject.

45. An infusion device comprising:
(i) a reservoir containing a first solution comprising an ammonium chloride salt and/or an amine and chloride ion;
(ii) a power source electrically connected to an electrode in contact with said solution and configured to produce N-chloro amine via electrolysis; and
(iii) a cannula in fluid communication with said solution for delivering said N-chloro amine to a subject.

46. An infusion device comprising:
(i) a first reservoir containing a first solution comprising an amine or a salt thereof;
(ii) a second reservoir containing a second solution comprising a chloride ions;
(iii) a power source electrically connected to an electrode in contact with said second solution and configured to produce hypochlorous acid or a salt thereof via electrolysis;
(iv) a mixing chamber for combining said first solution and said hypochlorous acid or a salt thereof to form an N-chloro amine; and
(v) a cannula in fluid communication with said mixing chamber for delivering said N-chloro amine to a subject.
